(19) United States
(12) Patent Application Publication
(10) Pub. No.: US 2019/0321429 A1
(43) Pub. Date: Oct. 24, 2019

(54) TOPICAL NATURAL LITHRAEA CAUSTICA EXTRACT COMPOSITION AND USE THEREOF IN TUMOURS

(71) Applicant: UNIVERSIDAD DE SANTIAGO DE CHILE, SANTIAGO (CL)

(72) Inventors: Sofia Antonieta MICHELSON QUINTANA, SANTIAGO (CL); Claudio Antonio ACUÑA CASTILLO, SANTIAGO (CL); Carolina Alejandra ROBLES CONTRERAS, SANTIAGO (CL); Javier MENA SILVA, SANTIAGO (CL)

(21) Appl. No.: 16/474,622

(22) PCT Filed: Dec. 26, 2017
(86) PCT No.: PCT/CL2017/050083

§ 371 Date: Jun. 28, 2019

(30) Foreign Application Priority Data
Dec. 29, 2016 (CL) 3396-2016

(51) Int. Cl. A61K 36/22; A61K 9/00; A61P 35/00

(52) U.S. Cl. CPC A61K 36/22; A61P 35/00; A61K 9/0014

(57) ABSTRACT

The invention relates to a natural composition for topical use, such as an ointment, comprising a Lithraea caustica extract and one or more pharmaceutically acceptable innocuous and non-toxic excipients, and to the use thereof in the treatment of benignant, premalignant and malignant solid tumors in the skin, including malignant melanoma. The extract is obtained via extraction with an organic solvent.

### DESCRIPTION

### Field of the Invention

The present invention is related to a composition of natural origin to topical use—such as ointment comprising an Lithrea caustica extract and one or more innocuous and pharmaceutically acceptable non-toxic excipients in the treatment of benign, pre-maligns and maligns solid tumors in the skin including malign melanoma. The extract is obtained by extraction with an organic solvent.

### State of the Art

Melanoma is a subtype of skin cancer which is the most aggressive derived from melanocytes. Its incidence has increased faster other types of cancer (from 3 to 7% yearly in Caucasian people), being the cancer with the biggest increase of incidence worldwide (IH® SER Cancer Statistics Factsheets: Melanoma Skin. National Cancer Institute. Accesed on April 2016 in: http://seer.cancer.gov/statfacts/html/melan.html ©WHO World Health Organization. Accessed on April 2016 at: http://www.who.int/uv/faq/skincancer/en/index1.html; Rebecca L. Siegel; Kimberly D. Miller, Cancer Statistics Ahmedin Jemal (2015), CA Cancer J Clin 2015; 65: 5-29Volume 65, Issue 1). Clinical staging of melanoma is based upon the infiltration of tumor cells in lymph nodes. When the disease is confined to the primary site resection surgical tumor generates a survival rate close to 100%. However, the greater the thickness and depth of melanoma at the primary site, the greater the likelihood of finding infiltration into lymph nodes or systemic level, and the prognosis is poorer. At this point surgical resection is no longer effective and even more this type of tumor is found to be highly resistant to conventional treatments such as chemo- and radiotherapy. It is due to this that it has begun to explore new strategies to cope with melanoma in advanced stages (http://www.bms.es/news/Documents/pdf/Entendiendo el melanoma FINAL12.pdf).

In Chile, cancer is the second leading cause of natural death after cardiovascular disease, however, in a projection for 2020, this will increase, becoming the leading cause of death. In Chile you have a record of about 300 new cases of melanoma a year, 122 deaths and nearly 1,000 patients prevalence (24.com © 2016. 24news, Rising UV radiation skin cancer prompts fears in Chile. Nov. 24, 2014. Accessed April 2016 at 14:57: http://www.news24.com/Green/News/Rising-UV-radiation-prompts-skin -cancer-fears-ini-Chile-20141124).

Currently, treatment options in advanced stages cost annually about 100,000 USD/patient, and offer on average less than seven months of survival. This cost and its impact economically depressed budgets of health systems. That is why there is growing public interest in the development, promotion and adoption of treatments for melanoma; safe, easy to apply and above all economic (© WHO 2016 World Health Skin Cancer They organized Accessed April 2016 at: http://who.int/faq/skincancer/en/index.html; Costhelper.Inc © 2016 Skin Cancer Treatment cost How Much Does skin Cancer Treatment cost?. Accessed in April 2016. (http://health.costhelper.com/skIn-cancer.html).

When the disease is confined to a primary site, where the disease began, the prognosis is good, the greater the thickness and depth of melanoma prognosis becomes poorer, implying that existing conventional treatments are ineffective. It is due to this that it has begun to explore new strategies to cope with melanoma in advanced stages.

Inmunotherapies emerge as a complement strategy for treating melanoma in advanced stages. Various strategies have been with which the immune system, among which is stimulated; Nonspecific immune stimulation, such as treatment with cytokines inmunostimulators as interleukin (IL-2) or interferon (IFN) α-2b; Inhibition of negative feedback of the immune system, where one example of this strategy is the Ipilimumab (Yervoy) drug, which is a monoclonal antibody blocking the action of CTLA-4 receptor and which results in preventing the “off” response of cytotoxic T lymphocytes, which are essential to the immune response anti-tumor (Franklin C, Livingstone E, Roesch a, B Schilling, Schadendorf D. Immunotherapy in melanoma: Recent advances and future directions. Eur J Surg Oncol. 2016 Sep 2. pii: S0748-7983 (16) 30866-6).

There have been a number of approaches have been used to induce an immune response against melanoma. One strategy is based on using the highest expression of melanoma associated antigens (AAM), on which could direct the immune system attack. Thus treatments have been developed that include active immunization against tumor, such as immunization of patients with irradiated tumors or tumor lysates antigen-specific adoptive transfer of own T lymphocytes (autologous) against tumor antigens or autologous dendritic cells loaded with tumor antigens (Engell-Noerregaard L, Hansen TH, Andersen MH, Thor Straten P, Svane IM Review of clinical studies on dendritic cell-based vaccination of Patients with malignant melanoma. Assessment of Correlation between clinical response and vaccine parameters. Cancer Immunol Immunother 2009 Jan; 58 (I): 1-14). In this last treatment, dendritic cell precursors from the patient's blood are obtained, which are differentiated in vitro and then they are cultured with lysates of cell lines. Melanoma and cytokines that induce their maturation, finally to be vaccinated patient. Initially R idolfi et al in 2006 (Ridolfi R, M Petrini, Fiammenghi L, M Stefanelli, Ridolfi L, M Ballardini, Migliore G, Riccobon A. Improved overall survival in dendritic cell vaccination-induced immunoreactive subgroup of advanced melanoma patients. J TRANSI Med 2006 Aug 16; 4:36) and subsequently the research group lysates aforementioned cell lines, reported that patients responding to immunotherapy, had responses delayed hypersensitivity (DTH) skin to apply a skin test with tumor antigens. This differed from the non-responders who showed no development of the immune reaction in the skin (Lopez MN., Pereda C, G. Segal, Muñoz L, Aguilera R., Gonzalez FE., Escobar A., A. Ginesta , Reyes D., Gonzalez R., A. Mendoza-Naranjo, Larrondo M., A. Compán Ferrada C, Salazar-Onfray F. (2009) Prolonged Survival of Dendritic Cell-Vaccinated Melanoma Patients Correlates With Tumor-Specific Delayed Type IV Hypersensitivity Response and Reduction of Tumor Growth Factor β-Expressing T Cells. J Clin Oncol 27, 945-952. Durán-Aniotz C, G. Segal, Salazar L, C Pereda, Falcon C, Volta F., R. Aguilera, Gonzalez R., Perez C, Tittarelli A., Catalan D., B. Nervi, Larrondo M., Salazar-Onfray F., Lopez MN. (2012). The immunological response and post-treatment survival of DC-vaccinated melanoma Patients are associated with an increased Th1/Th17 and reduced Th3 cytokine responses. Cancer Immunol Inmunother). The present invention produces a delayed hypersensitivity response (DTH) to apply the composition for topical use, such as an ointment, proposed (Martin SF., Esser PR., Weber FC, Jakob T., Freudenberg MA., Schmidt M. , Goebeler M. (2011). Mechanisms of chemical-induced innate immunity in allergic contact dermatitis. Allergy 66, 1152-1163). A difference of the above treatments, the present composition can be used as a minimally invasive treatment, which requires instrumental medical equipment and that produces an immune response and a long-term immunity against the tumor.

Other natral extracts as anti-cancer agents are also known in the prior art. For example, KR100251526 discloses the use of a compound based on urushiol and Rhus verniciflua extract as anticancer drug, antiviral and antioxidant agent and a method for preparing the extract. Rhus verniciflua extract contains one or more compounds selected from 3-pentadecylcatechol, 3-GBP 8′(Z)-pentadecenil catechol, 3-GBP 8′(Z), 11′ (Z)-pentadecadienil catechol and 3-GBP 8′ (Z), 11′ (Z), 13′-pentadecatrienil catechol of formula 4 obtained by extraction from the bark of Rhus verniciflua.

DE19854628 relates to the use of plant extracts of Anacardiaceae to prepare an extract for the treatment and prevention of tumors. Preferred are plants of the genus Rhus. The extract is obtained from fresh shoots, not yet lignified by alcoholic maceration. The extract is particularly suitable for the treatment of epithelial tumors, mesenchymal tumors and embryonal from undifferentiated tissue tumors. It is also suitable for the treatment of benign tumors such as adenoma, papilloma and/or polyps or malignant tumors such as carcinoma, mesenchymal tumor, and sarcoma.

JPS6479112 teaches a medicinal preparation comprising catechol butane which is useful for the treatment of benign lesions, premalignant and malignant solid tumors in the skin. The preparation is used for suppressing the proliferation of tumors.

CN101805246 relates to a medicinal preparation comprising an extracted from a traditional Chinese medicine phenolic lacquer. The preparation may be an oral formulation such as a tablet, capsule or the like, or injection. The preparation inhibits tumor activity and can be used for curing cancers such as leukemia, liver cancer, lung cancer, esophageal cancer, myeloma, breast cancer, colon cancer, prostate cancer and the like.

US2002001573 teaches a method for the treatment of cancerous tumors in mammals comprising administering an effective amount of an immunostimulating power and enhances the immune system of mammals and selectively attack and destroy cancer cells. The immunostimulator, preferred is urushiol, which is administered in a pharmaceutically acceptable carrier and may be co-administered in conjunction with other immunostimulators, radiation or other cytotoxic anti-cancer agents to improve its effect.

The present invention relates to a composition of natural origin for natural topical use may be an ointment. Such composition is useful for treating melanoma, and comprising a liter plant extract (Lithraea), obtained by an apolar organic extraction. The liter is a wild Chile plant, which grows naturally and is known to cause contact allergy (López, C, Kalergis, A., Becker, M., Garbarino, I, De loannes A. (1998). CD8+. T cells are the effectors of the contact dermatitis induced by urushiol in mice and are regulated by CD4+T cells Int Arch Allergy immunol 177: 194-201 Kalergis A., Lopez, C., Becker, M, Diaz, M., Sein, J, Garbarino, I, De loannes A. (1997) Modulation of fatty acid oxidation alters contact hypersensitivity to urushiols: Role of aliphatic chain beta-oxidation in processing and activation of urushiols. J Invest Dermatol 108: 57).

This naturally occurring composition for topical use has an effect on melanoma tumors in preclinical models, reducing tumor growth, and in some cases eliminating the tumor.

To date, it has developed a number of diseases associated with immunotherapy for melanoma clinical treatment strategies. They can be passive as the use of humanized antibodies against certain tumor antigens, which in melanoma have been successful. On the other hand the use of dendritic cells has been the strategy with better results and more widespread to date. This is to differentiate in vitro dendritic cells, from precursors obtained from peripheral blood, which are loaded with tumor antigens, whereupon these autologous cells are performed patients.

Other treatment applied against melanoma, compounds capable of generating haptens in proteins, activate the immune response nonspecifically triggering the induction of response against the tumor.

This naturally occurring composition for topical use has been created to be used continuously on the area where melanoma detected. It is important for successful treatment the patient does not exceed Stage III development, however it not means you cannot use it. In patients, the naturally occurring composition for topical use should be applied every 3-4 days. No more than that, because the idea is to be gradually absorbed by the skin and is constantly in touch.

From 70ths to date it described the use of dinitrochlorobenzene (DNCB) experimentally in the treatment of cancerous skin lesions such as melanoma. This effect has been shown to be due to induction of a hypersensitivity response delayed type (DTH), where the main component associated with this type of response is the production of a response (Th17 Karczmarczyk A, Karp M, Giannopoulos K. (2014). The role of Th17 cells in tumor immunity. Acta hematologic Polonica, Volume 45. pp 155-160. Yu Y., Hi Cho, D. Wang, K. Kaosaard, C. Anasetti, E. Celis, et al. Adoptive transfer of Tc1 cells elicits or Tc17 antitumor immunity against established through distinct melanoma Mechanisms. J Immunol, 190 (2013), pp. 1873-1881. Muranski P., et al. Tumor-specific Th17-polarized cells eradicate established large melanoma. Blood, 112 (2008), pp. 362-373). In these cases it described a wide range of results ranging from reversal in some cases to decrease metastatic processes other. Recently similar results have been described for treatment with diphencyprone (DCPC) which describe a regression in tumor growth, after being sensitized forearm (D. Buckley, Vivier A. (2001). The therapeutic use of topical contact sensitizers in dermatoses benign The British Journal of Dermatology 145, 385-405; Damian DL, Shannon KF, Saw RP, Thompson JF (2009) Treatment of metastatic cutaneous melanoma with extensive topical Australas J Dermatol diphencyprone 50, 266-271, Kim, Y. Topical diphencyprone as an Effective Treatment for Cutaneous Metastatic Melanoma Ann Dermatol 2012. 24, 373-375) the present invention then evaluates the cellular mechanisms associated aa induce an antitumor immune response and identifying cell types involved at least in the B16 murine melanoma model.

### BRIEF DESCRIPTION OF THE INVENTION

The present invention relates to a composition of natural origin for topical use—such as an ointment, comprising an extract of Lithraea caustic and one or more pharmaceutically acceptable safe and non-toxic excipients, and their use in the treatment of solid benign tumors, premalignant and malignant skin, including melanoma. In particular, its use in the treatment of malignant melanoma.

In particular, the present invention relates to a composition of natural origin for topical use may be an ointment. Such naturally occurring composition is useful for topical use to treat benign, premalignant and malignant solid tumors in the skin, including melanoma, and comprising a plant liter extract (Lithraea caustica), obtained by an apolar organic extraction. And said excipient is a harmless lipophilic excipient.

This naturally occurring composition for topical use has an effect on melanoma tumors in preclinical models, reducing tumor growth, and in some cases eliminating the tumor.

This naturally occurring composition for topical use has been prepared to be used continuously on the area where melanoma detected. It is important for successful treatment the patient does not exceed Stage III development, however, it does not mean you cannot use it. In patients, the naturally occurring composition for topical use should be applied every 3-4 days. No more than that, because it must be absorbed through the skin gradually and be in constant contact.

### BRIEF DESCRIPTION OF THE FIGURES

FIG. 1 Appearance Post Tumor Challenge DNCB.

FIG. 2 Tumor Growth without treatment and treatment with excipients and DNCB.

FIG. 3 Levels of T helper 1 cells untreated with excipients and DPCP.

FIG. 4 Treg lymphocytes untreated with excipients and DPCP.

FIG. 5 helper T17 without treatment and DPCP Excipients.

FIGS. 6A and 6B FIG. 6A Generation of specific lymphocytes against OA. 6B. Population growth CD8+.

FIG. 7A and 7B FIG. 6A Generation of specific lymphocytes against OA. 7B. Population growth CD8+.

FIGS. 8A and 8B FIG. 8A Treg cells in the spleen. FIG. 8B Treg cell populations.

FIGS. 9A and 9B FIG. 9A Th17 cells in the spleen. FIG. 9B lymphocyte Populations Th1.

FIG. 10 Th1 cells in the spleen.

FIGS. 11A and 11B FIG. 11A CD4 population. FIG. 11B CD4+CD8+and B cells in the spleen.

FIGS. 12A and 12B FIG. 12A CD8 population. FIG. 12B CD4+and CD8+cells in the tumor.

FIGS. 13A and 13B FIG. 13A splenocyte Proliferation CD4+cells. FIG. 13B Subpopulations CD4.

FIG. 14A and 14B FIG. 14A splenocyte Proliferation CD4+cells. FIG. 14B Subpopulations CD8.

FIG. 15 Effect of treatment on tumor growth.

FIG. 16 Survival Reasons

FIGS. 17A-17D FIGS. 17A dialing in tumor CD4+CD8 population. FIG. 17B dial tumor Population Tbet+. FIG. 17C dial tumor CD25Foxp3 population. FIG. 17D dial tumor Population RoRg+

FIG. 18 FIG. 18A dialing Population Spleen CD4+CD8+. FIG. 18B dial Spleen Population Tbet+. FIG. 18C dial Spleen Population CD25Foxp3. FIG. 18D dial Spleen Population RoRg+

### DETAILED DESCRIPTION OF THE INVENTION

The present invention relates to a composition of natural origin for topical use—such as an ointment, comprising an extract of Lithraea caustica (Liter) and one or more pharmaceutically acceptable safe and non-toxic excipients, and their use in the treatment of tumors solid benign, premalignant and malignant skin, including malignant melanoma. More preferably, the present composition of natural origin for topical ointment is made from an organic extract of plant Lithraea caustica (Liter), Chilean endemic plant. No visible effect immediately after application, but has an effect when used continuously over time in tumor growth and development of melanoma type. The composition for topical use comprising a Lithraea caustic extract (liter) and one or more pharmaceutically acceptable nontoxic excipients and safe. In particular, when the composition for topical use is an ointment, this presents a harmless lipophilic vehicle such as petrolatum or other vehicles of the same nature, which gives resistance, therefore, durability, and adsorbance long term the ointment on skin.

To extract preparation first proceeded to catalog the collected plant material, classifying by origin and size of the bush, season in which the plant material is collected, among other things, determined that in summer (December-April, southern hemisphere) it is the best time of extraction of plant material, and the preferred type of plant material were leaves of 5 cm and middle-aged (delimited between the new leaves growing at the tip, and the nearest to the trunk). Then, an organic extraction was performed, from sheets (which may be whole or ground previously) selected plant Lithraea caustica (Liter). For this purpose, an organic solvent was used in cold (particularly, petroleum ether), not to affect the composition of the extract. Subsequently, it filtered and the solvent evaporated using rotavapor controlling the temperature, and then allowed to dry at room temperature for 24 hrs.

This naturally occurring composition for topical use may be administered as a ointment, it is mainly applied in areas where surgery has not been effective, directly in the area where melanoma develops. Although it could also be used outside that area. This treatment also can be used as an adjunct to surgery treatment, avoiding tumor recurrence. For this treatment must be applied continuously, as determined doses (0.01 to 1% extraction v/v) two to three applications per week during the period in which the tumor is present, then a period preventive which may consist of 50% of weeks when treatment has been applied from the day begins to be applied until visibly disappears tumor.

The treatment of malignant melanoma, using DPCP which acts topical level, has been reevaluated in the literature area, HOMED two cases in which has achieved a significant regression process. Unfortunately the DCPC does not induce DTH responses in all individuals and DNCB if it does, but human use is prohibited. In order to evaluate the characteristics of both compounds and determine whether the mouse model may be a model extrapolated to humans, the effect of DPCP and DNCB in tumor growth to be subsequently compared to the effect of extract was evaluated liter.

To illustrate the advantages of the present invention has been made DPCP treatment, and extract DNCB liter in populations of rats, according to the examples below.

### EXAMPLE 1: TOPICAL TREATMENT DIPHENCYPRONE (DPCP)

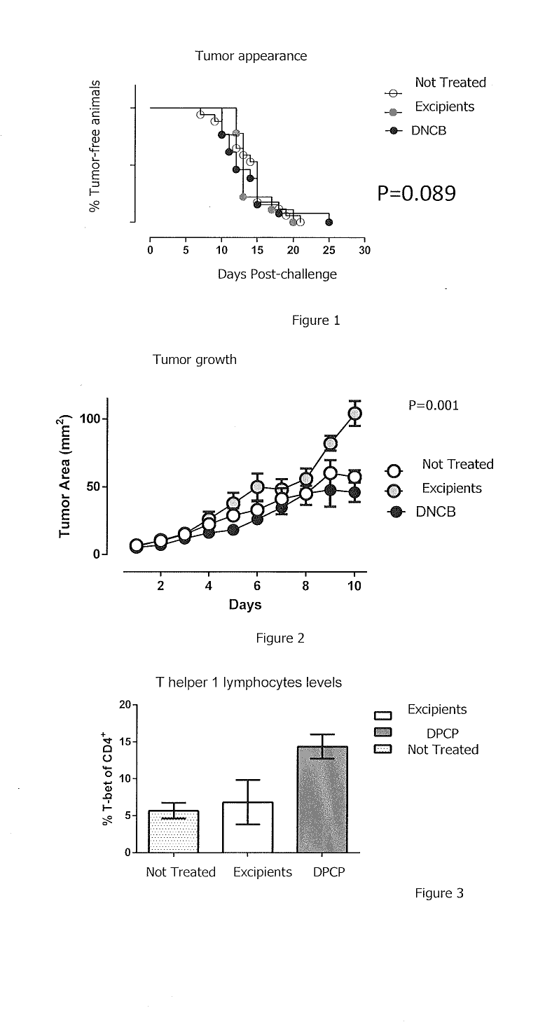

The effect of topical treatment with 0.1% diphencyprone in mice of the B6 strain transplanted tumor model was evaluated bl6. For which initially the animals were challenged with live cells reaching generating detectable tumors between 10 days until day 20 post challenge (FIG. 1). Tumor growth has a linear behavior reaching a peak at day 7 post near challenge (FIG. 2, black circles). Diphencyprone treatment induced a shift of the curve tumor appearance from an average detection of 13 to 16 days in treated animals, however, this change was not significant. Tumor growth when considering time 0 to the tumor mass was detected, shows no difference in the growth of control animals, treated with excipients, regarding treated DPCP (FIGS. 1 and 2, gray circles). For both cases the analysis of linear growth curves of both has no significant difference when comparing the slopes. Value p=0.85.

### EXAMPLE 2: EFFECT ON LYMPHOCYTE POPULATIONS

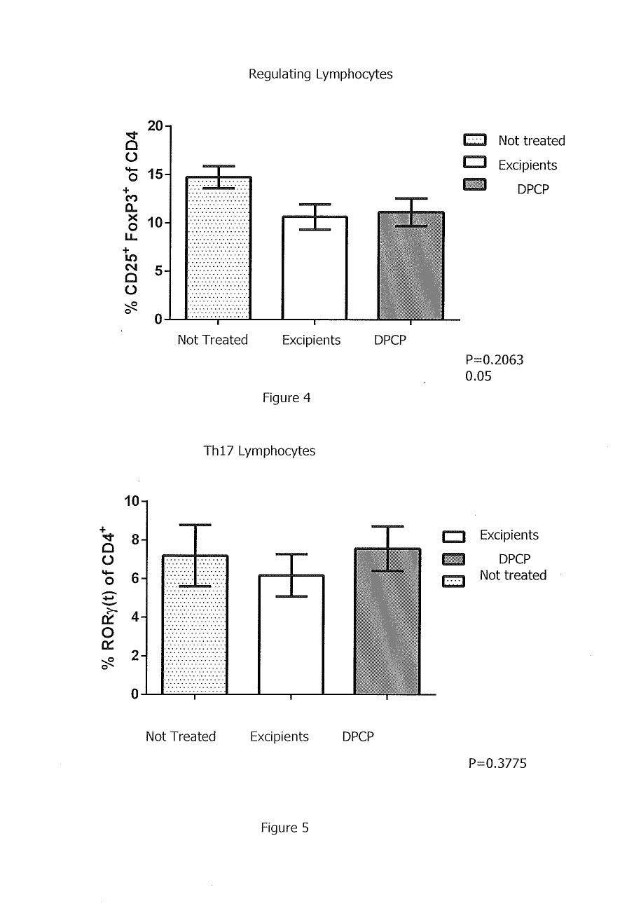

To analyze the effects of this treatment on lymphocyte populations T to determine if there was an analogy to these clinical cases effect, the treatment effect level spleen lymphocyte populations CD4 and CD8 was evaluated also subpopulations CD4 CD25 foxp3 which realizes regulatory T cells, the CD4+population Tbet+which realizes THL population and the presence of CD4+population+Ror range which realizes TH17 population. Treatment with DPCP associated tumor challenge, induced an increase in Th1 population present in spleen (FIG. 3). The analysis of mice treated b6 at least 8 experiment allows the characterization that this population increased compared to control animals, however, not be significant. Th17 population and the Treg population showed no change in the animals treated with excipients, with respect to control animals (FIGS. 4 and 5). No significant changes were observed in CD8 or CD4 populations associated with treatment (data not shown). Value p=0.59.

### EXAMPLE 3: SYSTEMIC RESPONSE AGAINST TUMOR CELLS

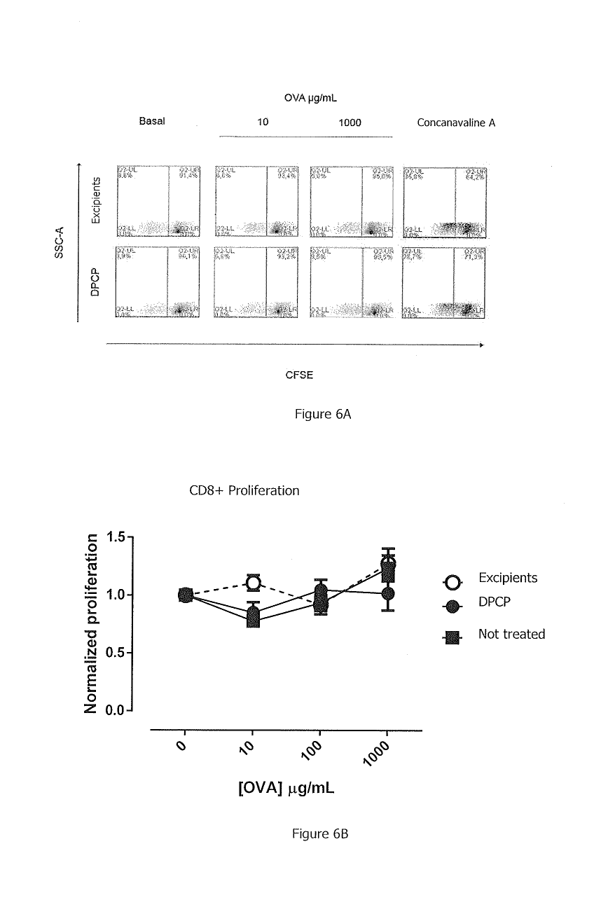

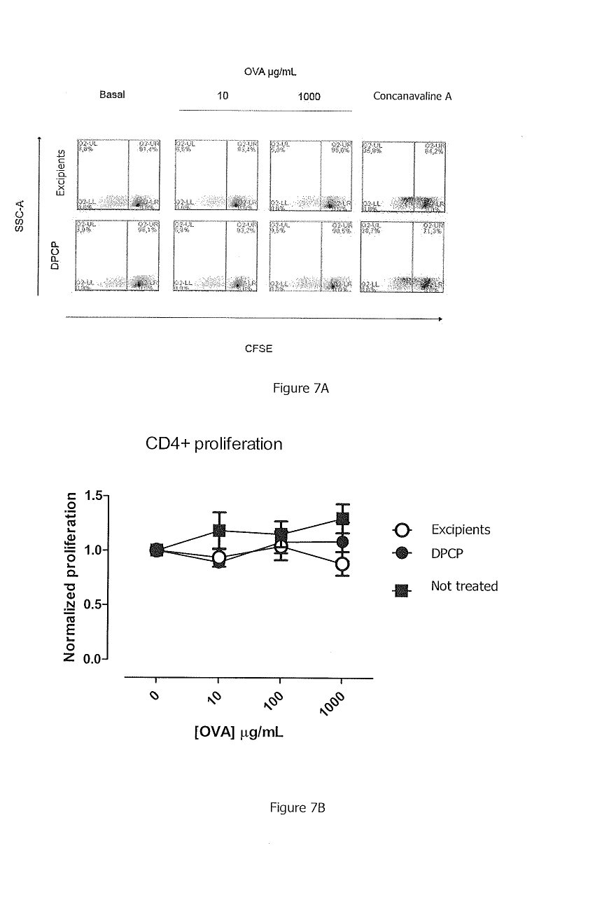

To assess whether a systemic response against tumor cells and a specific activation was induced, it was evaluated whether treatment with 0.1% to 1% DPCP induced generation of specific cell responses against OVA, for which the ability to induce proliferation was determined in culture by adding exogenous OVA, marking the splenocytes with the fluorescent marker carboxyfluorescein succinimidyl ester CSFE and marking CD4 or CD8 subpopulations with antibodies specific for these populations (FIGS. 6 and 7). The presence of positive populations that recognized OVA was not determined, but if non-specific proliferation was observed in response to concanavalin A. Together these results suggest that the curative treatment in mice with DPCP not have major effects on tumor model.

### EXAMPLE 4: EFFECT ON LYMPHOCYTE POPULATIONS DNCB

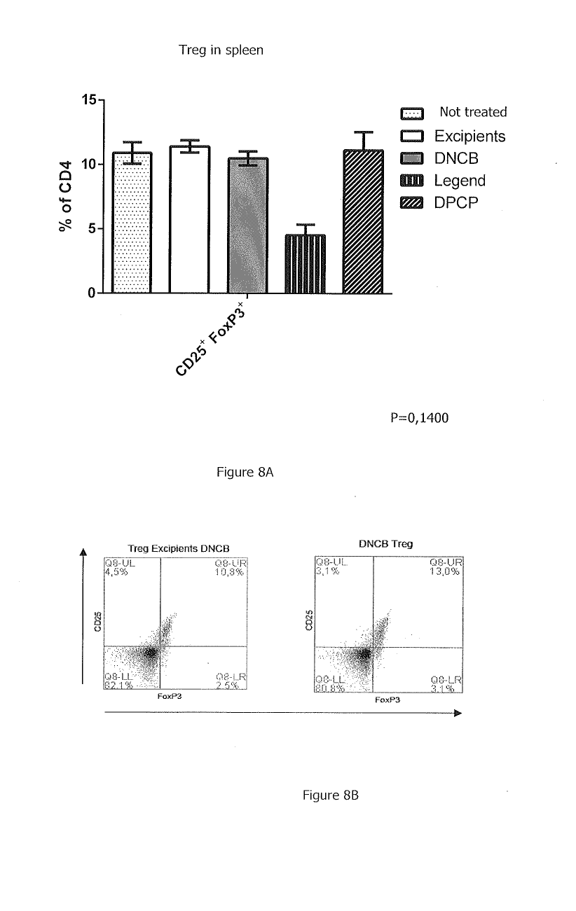

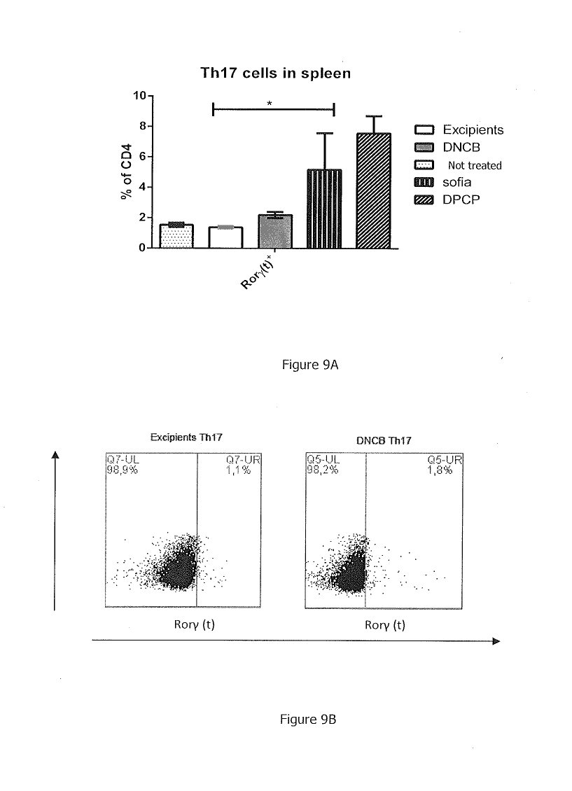

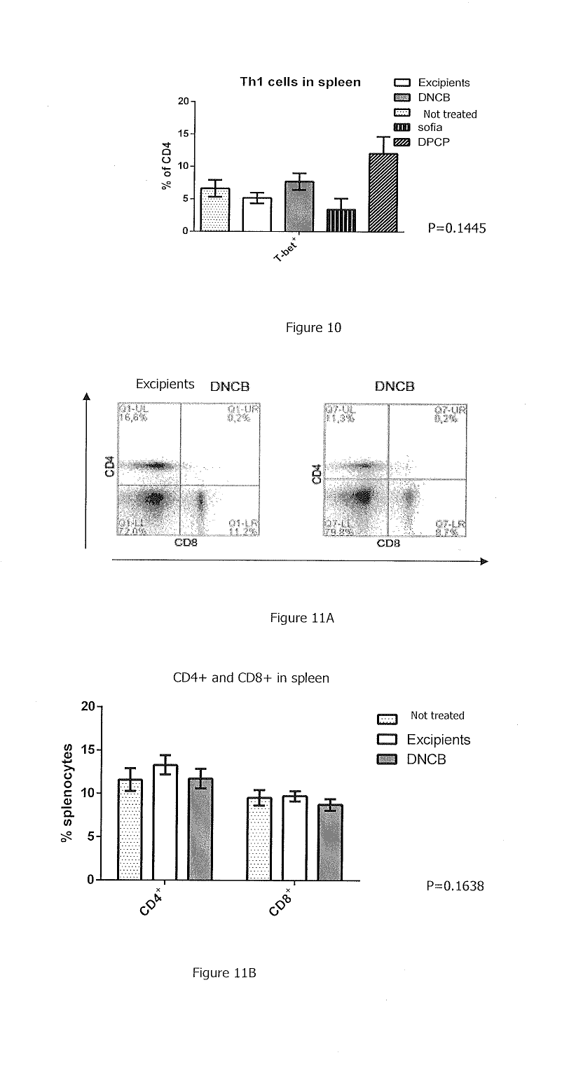

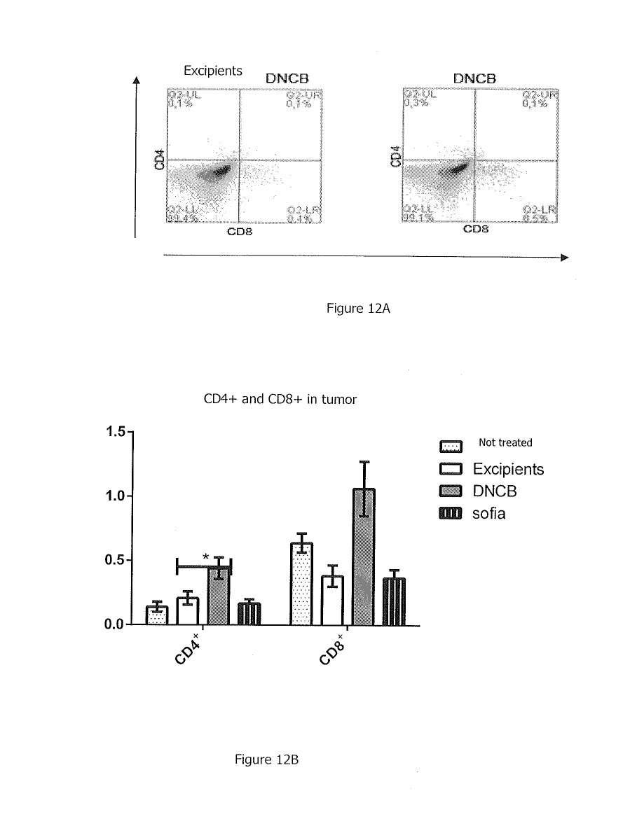

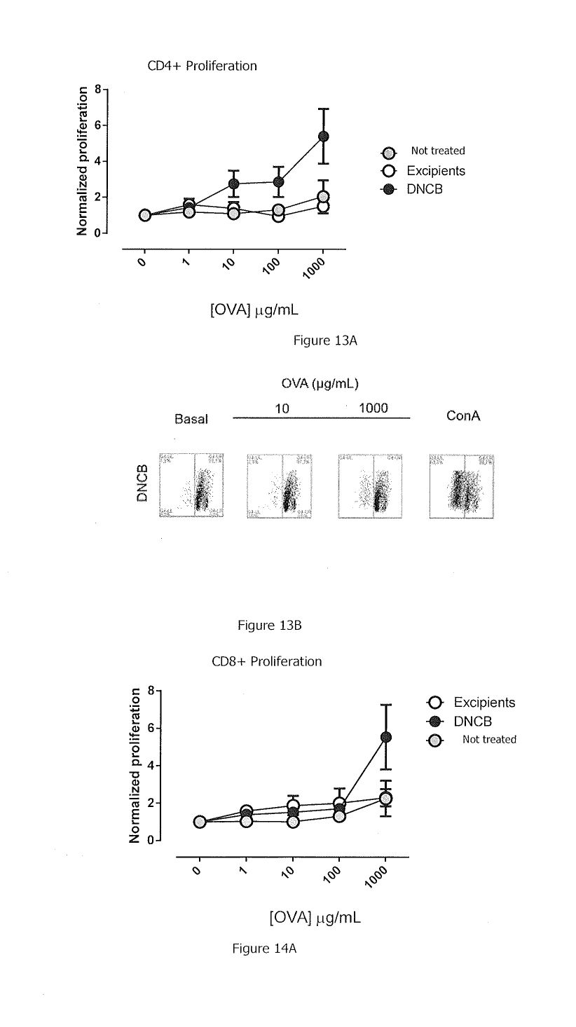

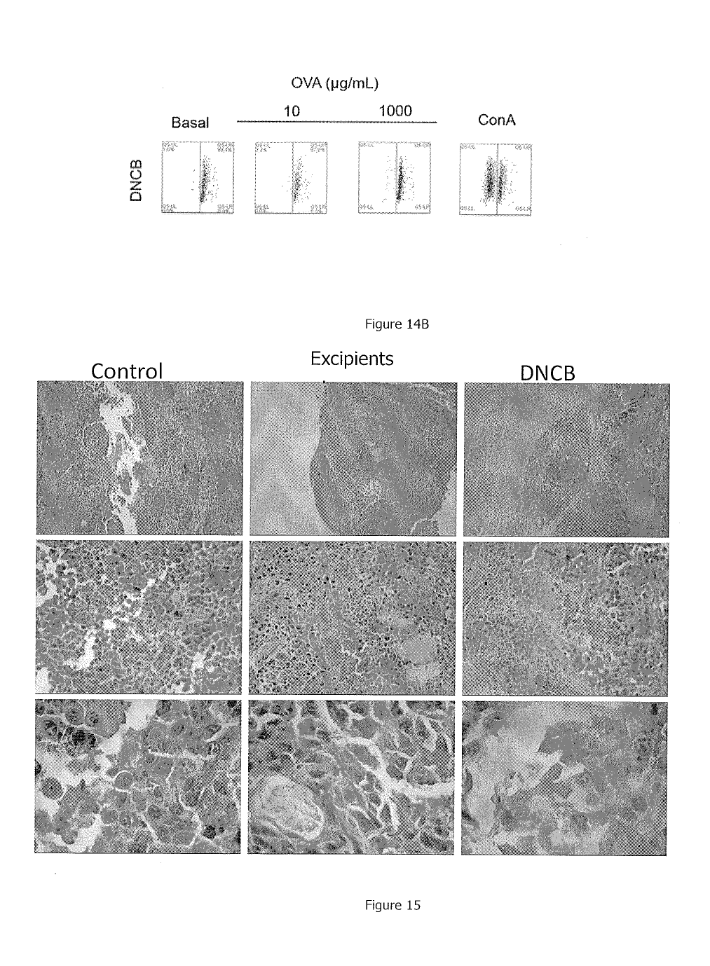

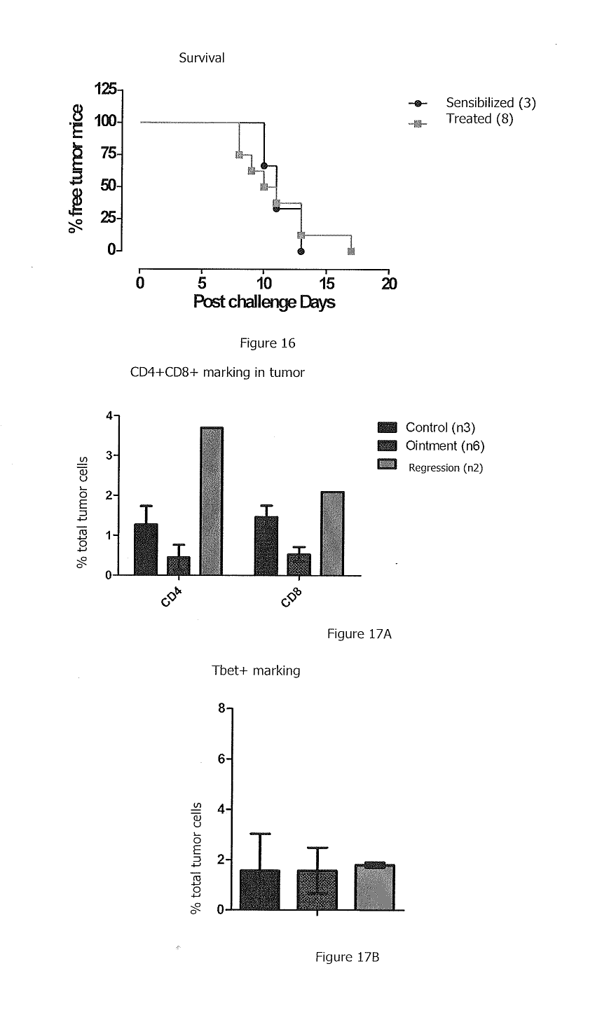

As for the previous case, it was determined that the effect of treatment, this time with DNCB on lymphocyte populations T as in previous case Treg (FIG. 8), Th1 (FIGS. 9A and 9B) and Th17 (FIG. 10) in b6 mice. As seen in the dot plot representing 9B, treatment associated tumor challenge, it induced an increase in the population Th17, measured as the CD4+Rory+population present in spleen. Analysis of at least 8 animals per treatment let characterize the increase in this population is significant. The Th1 population and the Treg population showed no change in the animals treated with excipients, with respect to control animals. Thus either CD4 populations, or CD8 (FIGS. 11 and 12) showed significant changes associated with the treatment, but was observed in both cases an increase in the levels of lymphocyte infiltrating, both CD4+and CD8+, which could account for the delay observed in tumor growth.

### EXAMPLE 5: SYSTEMIC RESPONSE TO TUMOR CELLS

To assess whether a systemic response against tumor cells and a specific activation was induced, the presence of lymphocytes specific against OVA was evaluated, the ability to induce proliferation in culture by the addition of exogenous OVA labeling the splenocytes with CSFE and marking was determined subpopulations CD4 or CD8 (FIGS. 13 and 14). As seen, in case there was a consistent CD4 proliferative response to all concentrations tested, unlike what was observed for CD8 where only the highest concentration of OVA induced an increase in the proliferation of this specific population. The presence of positive populations that recognized OVA was not determined, but if non-specific proliferation was observed in response to concanavalin

A.

### EXAMPLE 6 ANIMALS Treated WITH EXTRACT LITHREOL

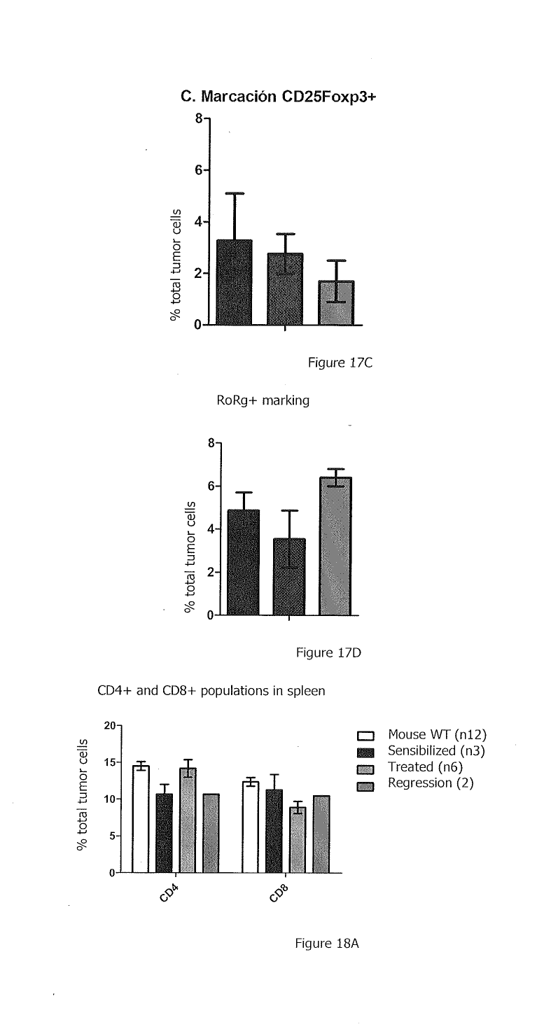

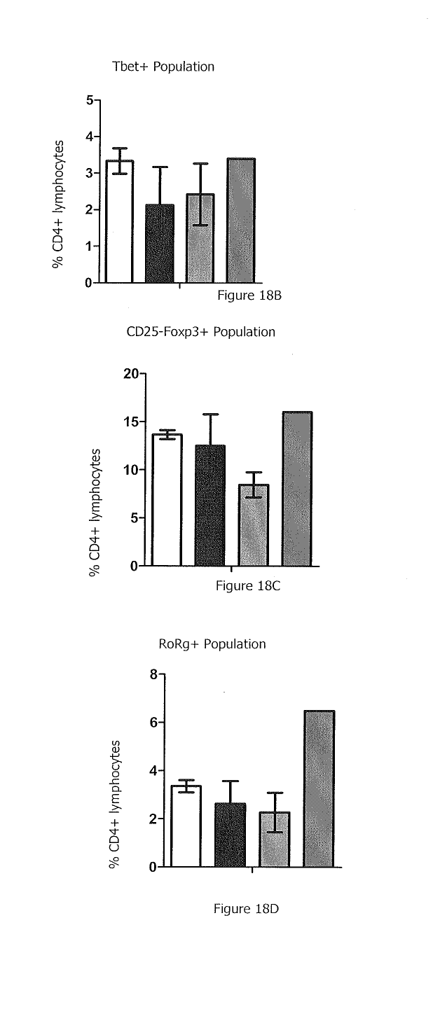

Finally, for the case of animals treated with liter extract, it was observed that tumor level, two behaviors that may indicate a temporary dynamic behavior of cell populations response to tumor level. On one hand, there are subjects exhibiting an apparent regression of tumor growth, at this stage high levels of populations marking for CD8 and CD4 were observed, well above controls Unlike in the most advanced stage where the tumor manages return to normal growth, a decrease of these markings appreciated, under control, so that there was possibly a mechanism by which this reduction would help tumor growth (FIG. 17A). Furthermore, the bearings of the other populations are inconclusive (FIG. 17B, 17C and 17D). With respect to systemic level measurements markings populations above mention in spleen, they showed a difference of the animals treated compared to controls, so the treatment appears to decrease the CD8+population, and increasing CD4+populations level systemic (FIG. 18A), the latter, when analyzing the CD4 positive subpopulations, a tendency to decrease in regulatory T cell population (FIG. 18B) the increase would be associated with a poor prognosis was observed. Moreover, animals with regression showed no observable changes in the population of CD4 and CD8 in spleen, no clutch to analyze subpopulations was a clear increase Th17 population (FIG. 18C) population has had a controversial role in cancer, however, in that case could help an inflammatory response to the tumor.

What is claimed:

1. Composition of natural origin for topical use, comprising a pharmaceutically acceptable extract obtained from extraction with organic solvents from a plant Lithrea caustica (liter) or one of its parts and one or more innocuous and non-toxic excipients.

2. The composition of natural origin for topical use of claim 1, being an ointment.

3. The composition of natural origin for topical use, wherein said part of the plant leaf is liter.

4. The composition of natural origin for topical use of claim 1, wherein said extract with organic solvents is an apolar organic extract.

5. The composition of natural origin for topical use of claim 2, wherein said carrier is a lipophilic innocuous excipient.

6. Use of the composition of natural origin for topical use of claim 1, wherein serves to prepare a medicament useful to reduce or eliminate tumor growth melanoma tumor type medication.

7. Use of the composition of natural origin for topical use of claim 1, used to prepare a medicament useful in the treatment of solid tumors benign, premalignant and malignant skin.

8. Use of the natural composition for topical use of claim 7, used to prepare a medicament useful in the treatment of malignant melanoma.

9. Method for reducing tumor growth or remove a melanoma tumor type, comprising applying to the area or near the area where melanoma has been detected, a composition for topical use comprising an extract obtained by extraction by organic solvents liter plant or portion thereof, and one or more innocuous excipients and non-toxic pharmaceutically acceptable.

10. Method for treating solid tumors benign, premalignant and malignant skin, comprising applying to the area or near the area where detected melanoma, a natural composition for topical use comprising an extract obtained from an organic solvent extraction plant liter or one of its parts, and one or more safe and non-toxic, pharmaceutically acceptable excipients.

11. The method of claim 10, comprising applying during the period in which the tumor is present and the area or near the area where detected melanoma, a natural composition for topical use, two to three times in the week, in an area of 2.5 cm², said composition comprising 0.01 to 1% extraction v/v, and then applying said composition to a preventive period may consist of half of the previous period, until visibly removes the tumor.

12. Method for treating solid tumors benign, premalignant and malignant skin, comprising applying to the area or near the area where surgery was performed infective, a natural composition for topical use comprising an extract obtained from an organic solvent extraction plant liter or one of its parts, and one or more safe and non-toxic pharmaceutically acceptable excipients.

13. A method for treating complementarily after surgery for melanoma, which comprises applying to the area or near the area where surgery, a natural composition for topical use comprising an extract obtained by extraction by organic solvents was performed liter plant or portion thereof, and one or more innocuous and non-toxic pharmaceutically acceptable excipients.

14. Method to prevent recurrence of a malignant tumor malignant, premalignant or skin, comprising applying to the area or near the area where surgery was performed, a natural composition for topical use comprising an extract obtained from an organic solvent extraction plant liter or one of its parts, and one or more safe and non-toxic pharmaceutically acceptable excipients
